(19) United States
(12) Patent Application Publication
(10) Pub. No.: US 2026/0053662 A1
(43) Pub. Date: Feb. 26, 2026

(54) APPARATUS AND METHOD FOR DRY CRYOTHERAPY

(71) Applicant: JTL Enterprises, Inc., Clearwater, FL (US)

(72) Inventors: Kevin CONAWAY, Tampa, FL (US); Paul LUNTER, Palm Harbor, FL (US); Timothy ELLIOT, Pinellas Park, FL (US)

(21) Appl. No.: 19/304,348

(22) Filed: Aug. 19, 2025

Related U.S. Application Data
(60) Provisional application No. 63/685,002, filed on Aug. 20, 2024.

(51) Int. Cl. A61F 7/00

(52) U.S. Cl. CPC A61F 7/0053; A61F 7/0085; A61F 2007/0024; A61F 2007/003; A61F 2007/0039; A61F 2007/0056; A61F 2007/0095

(57) ABSTRACT

A dry cryotherapy apparatus for use by a user. The apparatus according to this aspect comprises a support structure defining a user receiving area at which the user will be located for dry cryotherapy. A plurality of liquid bladders are located in the user receiving area, the bladders being configured to contain a quantity of liquid. A reservoir (e.g., a water tank) operative to contain a quantity of liquid is also provided, the reservoir being in selective fluid communication with the plurality of bladders via a liquid circulation arrangement. The apparatus further includes a cooler operative to cool the liquid in the reservoir to a selected temperature. Supply of the liquid from the reservoir to one of the liquid bladders causes the bladder to provide therapeutic engagement with an adjacent portion of the user's body. The combination of liquid and gas bladders are a more technological-based approach as compared to sitting on top of a cold waterbed, resulting in an automated mechanical system with water bladders and gas bladders that sync together and are automatically adjusted based upon user weight/size and desired experience in a reclined position.

### CROSS-REFERENCE TO A RELATED APPLICATION

This application is based upon and claims the benefit of U.S. provisional application Ser. No. 63/685,002, filed Aug. 20, 2024, which is incorporated herein by reference in its entirety for all purposes.

### FIELD OF THE INVENTION

The present invention relates generally to devices for providing cryotherapy (i.e., cold plunge therapy).

### BACKGROUND OF THE INVENTION

Cold hydrotherapy has been utilized by physical therapists and athletic trainers for many years. “Cryotherapy literally means cold therapy . . . . Cold therapy is the ‘I’ part of R.I.C.E. (rest, ice, compression, and elevation). This is a treatment recommended for the home care of many injuries. It is often a suggestion for sports injuries . . . . Experts believe that cold therapy can reduce swelling that is tied to pain. It may also reduce sensitivity to pain.” https://www.urmc.rochester.edu/encyclopedia/content.aspx?contenttypeid=134&contentid=95.

In recent years, there has been a noticeable shift in consumer awareness and demand for various forms of wellness, self-care, and post-workout recovery. According to Mckinsey, “[e]ighty-two percent of U.S. consumers now consider wellness a top or important priority in their everyday lives.” https://www.mckinsey.com/industries/consumer-packaged-goods/our-insights/the-trends-defining-the-1-point-8-trillion-dollar-global-wellness-market-in-2024.

Beyond athletes, various forms of cold therapy (cold plungers, cryotherapy chambers, etc.) have also gained popularity among the wellness community. However, most forms of cold therapy are not efficient for high-volume commercial settings, as the user is required to get undressed and wet. There are also sanitary concerns (e.g., sweaty athletes getting in the same small cold pool) and safety concerns (e.g., slip and fall on wet floor) associated with various forms of cold therapy.

The present invention recognizes and addresses considerations of prior art constructions and methods.

### SUMMARY OF THE INVENTION

One aspect of the present invention provides a dry cryotherapy apparatus for use by a user. The apparatus according to this aspect comprises a support structure defining a user receiving area at which the user will be located for dry cryotherapy. A plurality of liquid bladders are located in the user receiving area, the liquid bladders being configured to contain a quantity of liquid such as water. A reservoir (e.g., a water tank) operative to contain a quantity of the liquid is also provided, the reservoir being in selective fluid communication with the plurality of bladders via a liquid circulation arrangement. The apparatus further includes a cooler operative to cool the liquid in the reservoir to a selected temperature. Supply of the liquid from the reservoir to one of the liquid bladders causes the bladder to fill such that an adjacent portion of the user's body will be cooled by the liquid.

According to some exemplary embodiments, the cooler may be external to the reservoir such that the liquid being cooled circulates from the reservoir through the cooler and back to the reservoir.

According to some exemplary embodiments, at least one pump may be provided to supply the cooled liquid to the liquid bladders. In addition, at least one temperature sensor and at least one pressure sensor may be provided. A plurality of electromechanical valves may also be situated in the liquid circulation arrangement. In such embodiments, a controller may be programmed to effect operation of the at least one pump and the electromechanical valves based at least in part on data from the at least one temperature sensor and the at least one pressure sensor.

According to some exemplary embodiments, a movement mechanism may be associated with at least some of the liquid bladders, the movement mechanism being operative to cause an associated liquid bladder to cover a front part of the adjacent portion of the user's body. For example, the movement mechanism may comprise a gas bladder associated with the associated liquid bladder. In such embodiments, a compressed air source operative to inflate the gas bladder may be provided.

According to some exemplary embodiments, the plurality of liquid bladders comprises at least four bladders, at least five bladders, or at least six bladders.

According to some exemplary embodiments, the plurality of liquid bladders may comprise at least one shoulder bladder, at least one back bladder, at least one seat bladder, a pair of arm compression bladders, a pair of side compression bladders, and a pair of leg compression bladders.

According to some exemplary embodiments, the dry cryotherapy apparatus further comprises a user interface, such as a touch screen.

Another aspect of the present invention provides a dry cryotherapy apparatus for use by a user. The apparatus comprises a support structure defining a user receiving area at which the user will be located for dry cryotherapy. A user interface is also provided. A plurality of liquid bladders are located in the user receiving area, the liquid bladders configured to contain a quantity of liquid. A reservoir operative to contain a quantity of the liquid is also provided, the reservoir being in selective fluid communication with the plurality of bladders via a liquid circulation arrangement. At least one pump is located in the liquid circulation arrangement to supply the liquid from the reservoir to the liquid bladders. A plurality of electromechanical valves are also situated in the liquid circulation arrangement. Also provided is a cooler operative to cool the liquid in the reservoir to a selected temperature. Supply of the liquid from the reservoir to one of the liquid bladders causes the bladder to be in therapeutic engagement with an adjacent portion of the user's body.

A further aspect of the present invention provides a method of performing dry cryotherapy. One step of the method involves providing an apparatus having a support structure defining a user receiving area at which the user will be located for dry cryotherapy and a plurality of liquid bladders located in the user receiving area, the liquid bladders being in a deflated state. Another step of the method involves providing a source of cold water. Another step of the method involves supplying a quantity of the cold water to the plurality of liquid bladders so that the bladders will be in an inflated state. Another step of the method involves causing the quantity of water to leave the bladders so that the bladders are again in the deflated state.

According to some exemplary methodology, the step of providing a supply of cold water involves chilling the water to a temperature of between approximately 33 to 45 degrees Fahrenheit. Moreover, the bladders may be maintained in the inflated state for a predetermined period of time between approximately 8-10 minutes.

The accompanying drawings, which are incorporated in and constitute a part of this specification, illustrate one or more embodiments of the disclosure and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended drawings, in which:

FIGS. 1A and 1B are perspective views of apparatus in accordance with embodiments of the present invention.

FIG. 2 is a perspective view of an apparatus in accordance with an embodiment of the present invention showing various liquid bladders (or “water bags”) that may be provided.

FIGS. 3A and 3B show the apparatus of FIG. 2, occupied by a user with the liquid bladders, in the deflated and inflated states, respectively.

FIGS. 4A and 4B are fragmentary views of FIGS. 3A and 3B, respectively, showing the shoulder water bag in the deflated and inflated states.

FIG. 5 shows the apparatus of FIG. 2 showing the back water bag with other bags removed for purposes of illustration.

FIG. 6A is a fragmentary view of the apparatus of FIG. 2 showing the seat water bag in the deflated state.

FIGS. 6B and 6C are views similar to FIG. 6A (with no user and a user present, respectively) showing the seat water bag in the inflated state.

FIGS. 7A and 7B are fragmentary views of FIGS. 3A and 3B, respectively, showing the leg compression water bags in the deflated and inflated states.

FIGS. 8A and 8B are fragmentary views of FIGS. 3A and 3B, respectively, showing one of the arm compression water bags in the deflated and inflated states.

FIGS. 9A and 9B are fragmentary views showing a side compression water bag in the deflated and inflated states, respectively.

FIG. 10 is a diagrammatic representation showing components of a dry cryotherapy apparatus in accordance with an embodiment of the present invention.

FIG. 11 is a diagrammatic representation of a dry cryotherapy apparatus intended to support the user in a reclined position with a pivotal leg support portion to facilitate ingress and egress by the user.

FIGS. 12-14 show additional aspects of the embodiment of FIG. 1A.

Repeat use of reference characters in the present specification and drawings is intended to represent same or analogous features or elements of the invention according to the disclosure.

### DETAILED DESCRIPTION OF PREFERRED EMBODIMENTS

Reference will now be made in detail to presently preferred embodiments of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents.

An apparatus according to preferred embodiments of the present invention provides extreme cold therapy similar to a cold plunge except the user remains clothed and dry during the session. Most of the user's body is exposed to very cold water separated from the body by a flexible bladder that conforms to the shape of the body. Typically, the water will be at a selected temperature in the range of between approximately 33-45° F., such as approximately 40° F. One skilled in the art will appreciate that other temperatures may be selected in some cases. For example, the liquid may be cooled to a temperature below 32° F. in some cases. If water is the liquid, then in may be necessary in this situation to add antifreeze to the water. The apparatus is preferably configured so that the bladder will conform to the shape of the body despite variances in the size of one person's body to another.

Preferably, the user will sit in a semi-reclined or reclined ergonomic position for the session. For example, the duration of the session may be between 3-10 minutes, such as about 3-5 minutes for users who prefer shorter sessions or about 8-10 minutes for users who prefer longer sessions, as desired. In some embodiments, the user may initially be in a more upright seated position, but the seating structure may recline before the cold therapy session begins. Multiple separate bladders are preferably provided, such as behind the user's neck, back, legs, and sides. In some cases, bladders may be provided for the front of the body as well. These may be mounted on movable structures that allow the bladders to be away from the body for ingress and egress, but that move into contact with the front of the body for the session. The movable structures (e.g., pivotal arms) may be moved by hand or in some cases by suitable actuators (e.g., electric, pneumatic, etc.). In some embodiments, the movable structure may be in the form of inflatable gas bladders as described below.

According to various preferred embodiments, a computer-controlled cooling system is provided to maintain water at the selected temperature in a holding tank (e.g., a tank in the range of 10-40 gallons, such as a tank in the range of 20-40 gallons) between sessions. A series of pumps, valves, solenoids, sensors (e.g., pressure and temperature sensors), and piping is designed to pump cold water (or other suitable liquid) into the various liquid (e.g., water) bladders to maintain the cold water in close proximity to the user's body during the session. Preferably, sufficient water is pumped into the bladders under the user so that the user will essentially “float” on top of the water during the session. In other words, the balance of air pressure in the air bladders below the water bladders and the water pressure in the water bladders provides a feeling of floating while still sinking in to get maximum benefit. The relaxation benefits of the “floating” sensation further enhances the experience. The movable mechanism, an example of which is further described below, may support each bladder to gently but thoroughly press the bladder against the user to deliver maximum cold exposure over the body's surface.

An important aspect of preferred embodiments—and the entire user experience of wrapping or enveloping the user in cold—is the implementation of technology combining the water bladders and gas bladders. This provides a superior technological approach as compared to sitting on top of a cold waterbed. The result is an automated mechanical system with water bladders and gas bladders that sync together and are automatically adjusted based upon user weight/size and desired experience in a reclined position.

It will be appreciated that a suitable user interface, such as a touchscreen, is preferably provided so that the user can control various functions of the apparatus and/or view entertainment during the session. For example, the user is preferably able to stop the session at any time. In addition, activation of the apparatus may be controlled electronically from a front desk, barcode, RFID, and/or other methods (e.g., to limit usage to eligible customers or members only).

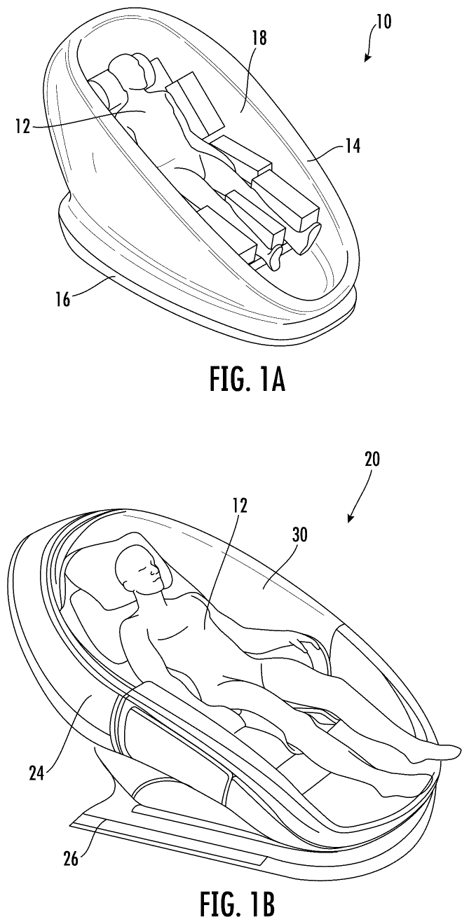

FIG. 1A shows a first example of an apparatus 10 for providing dry cryotherapy to a user 12 in accordance with an embodiment of the present invention. As can be seen, apparatus 10 has a housing 14 with a base portion 16 for resting the apparatus on a surface (floor). As one skilled in the art will appreciate, housing 14 (and the housings of the other embodiments described below) will contain various other components of apparatus 10 and will include a suitable frame arrangement to provide support and rigidity to the overall structure. As used herein, the term “support structure” may be considered to be the housing, the frame, or portions of the housing and/or frame. Housing 14 further has a user receiving area 18 where the user 12 is located for the session. In this case, the user receiving area 18 is configured so that, at least initially, the user 12 will be in a seated position. Embodiments are contemplated in which the user receiving area 18 can further recline for the cold therapy session. As will be explained further below, the liquid bladders are located in the user receiving area 18 to be or to move into therapeutic contact with the body of user 12.

FIG. 1B illustrates a second example of an apparatus 20 for providing dry cryotherapy to user 12 in accordance with an embodiment of the present invention. Apparatus 20 has a housing 24 with a base portion 26 for supporting the apparatus on a surface (floor). Housing 24 further has a user receiving area 28 where the user 12 is located for the session. In this case, the user receiving area 28 is configured so that the user 12 will be in a reclined position. The liquid bladders are located in the user receiving area 28 to be or be moved into therapeutic contact with the body of user 12. In this embodiment, a cover 30 is provided to at least partially enclose the user 12 during the session. For example, the cover 30 may be pivoted at one end to allow ingress and egress of the user or may have a sufficient opening for passage of the user. In any event, cover 30 creates more of a private space than the previous embodiment, which some users may prefer. Preferably, apparatus 20 includes a pivotable leg rest that moves down to facilitate ingress but moves up so the legs are slightly raised during the session.

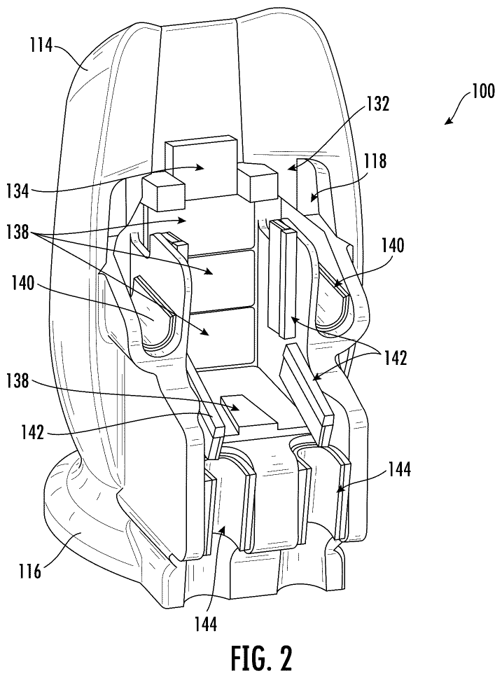

A further example of an apparatus 100 in accordance with an embodiment of the present invention is illustrated in FIG. 2. As can be seen, apparatus 100 has a housing 114 with a base portion 116 for supporting the apparatus on a surface (floor). Housing 114 further has a user receiving area 118 where the user 12 is located for the session. In this case, the user receiving area 118 is configured so that, at least initially, the user 12 will be in a seated position.

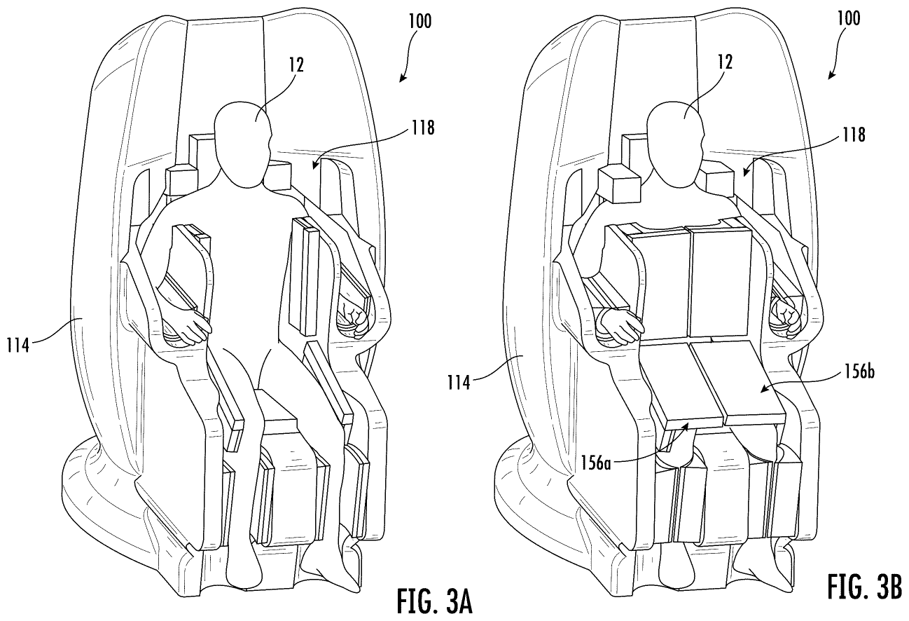

Various liquid bladders (or “water bags”) are located in the user receiving area 118 to expand and/or otherwise inflate into contact with the body of user 12. As can be seen, this embodiment has shoulder bladders 132 and an associated bladder 134 for the back of the head. A series of back water bladders 136 are located behind the user's back. One or more seat bladders 138 are located in the area under a seated user's buttocks. Arm compression bags 140, side compression bags, 142, and leg compression bags 144 are also provided. FIGS. 3A and 3B show the user 12 located in the user receiving area 118 with the bladders in a deflated state for ingress and egress and the inflated state for use during a dry cryotherapy session, respectively.

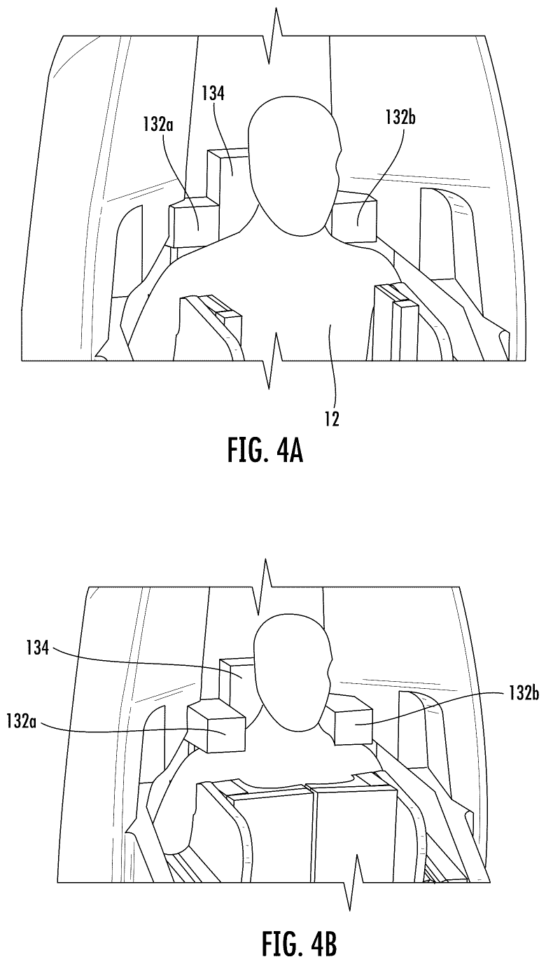

The various bladders will now be discussed in greater detail. Turning first to FIGS. 4A and 4B, the shoulder bladders 132 and head bladder 134 can be seen. In this embodiment, a pair of shoulder bladders 132a and 132b are provided on right and left sides of the head of user 12. In the deflated state (FIG. 4A), the bladders 132a and 132b are retracted so as to be almost entirely behind the user. In the inflated state (FIG. 4B), the bladders 132a and 132b extend forward so as to be on top of the user's shoulders. The head bladder 134 is similarly inflated to move the user's head slightly away from the hard surface of the housing 114.

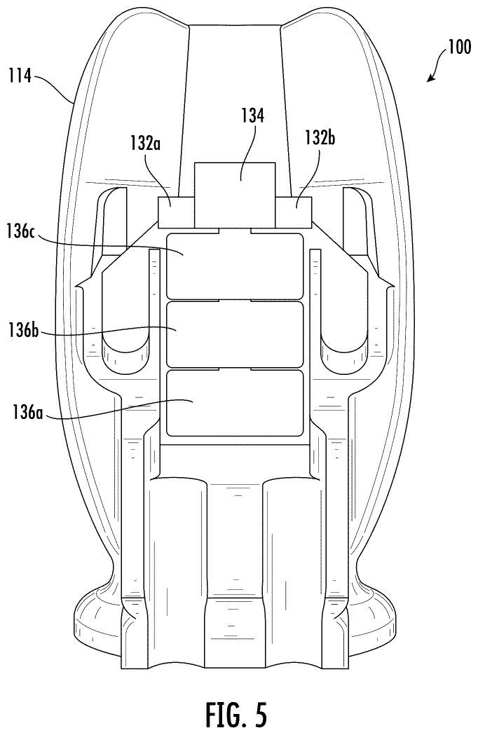

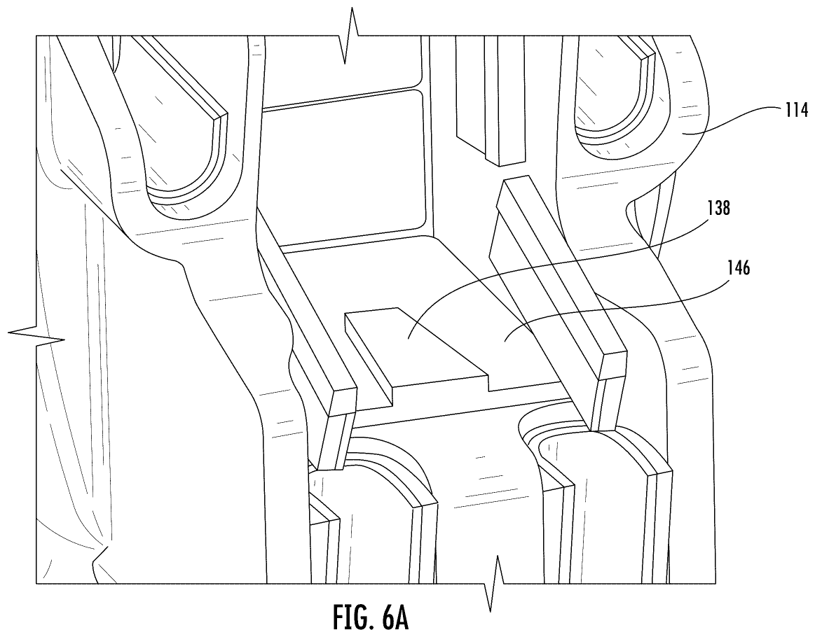

Back bladders 136 are most clearly shown in FIG. 5. In this case, three back bladders 136a-c are provided at the lower back, midback, and upper back positions, respectively. Preferably, bladders 136a-c may slightly overlap to create substantially continuous coverage along the entirety of the user's back. Inflation of the bladders 136a-c preferably causes the user's back to move slightly away from the hard underlying surface of housing 114.

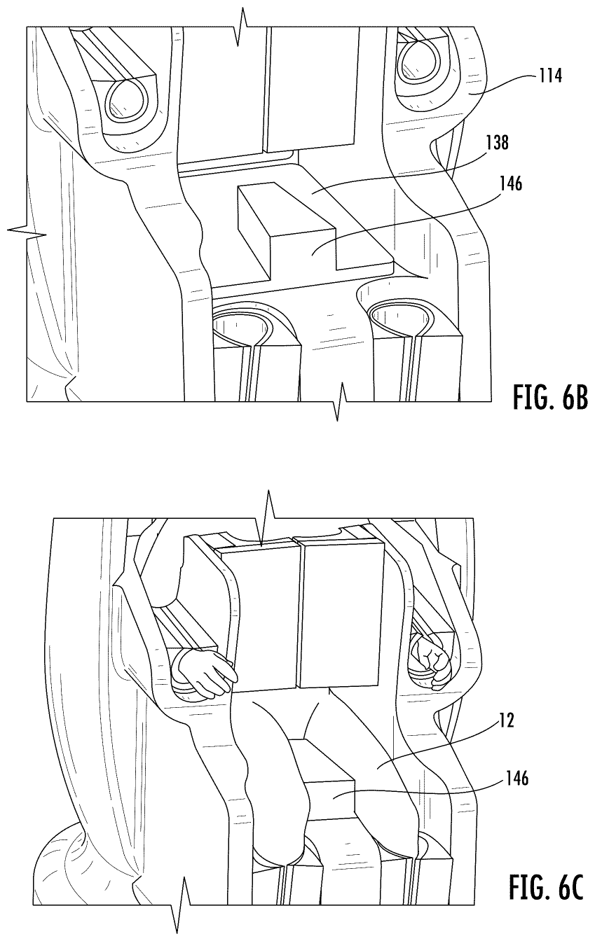

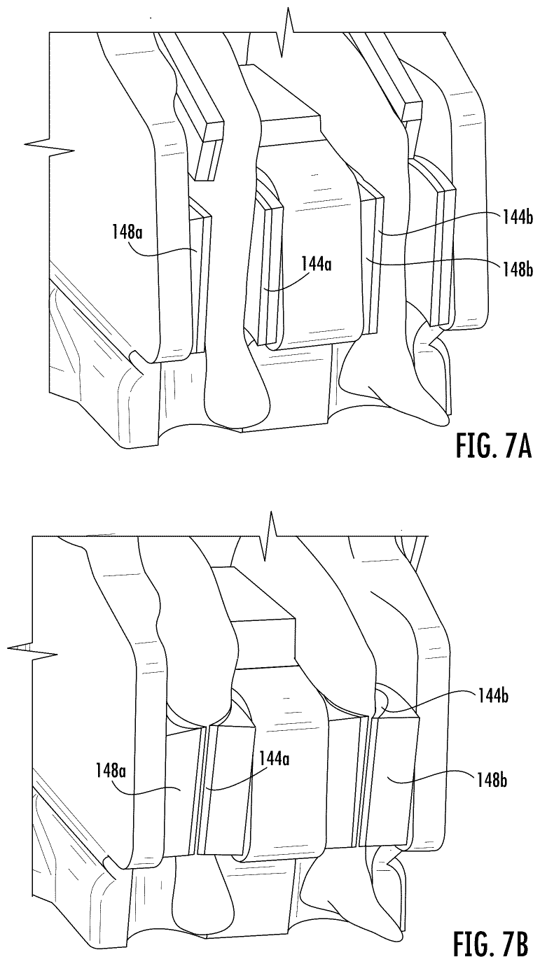

FIGS. 6A and 6B illustrate seat bladder 138 in deflated and inflated states, respectively. Although multiple separate bladders may be used to comprise seat bladder 138, the present embodiment uses a single bladder for this purpose. As can be seen, however, seat bladder 138 has a forward trapezoidal portion 146 which rises when seat bladder 138 is inflated. This provides cooling therapy to the inside of the user's thighs, as shown in FIG. 6C. As with other bladders described above, seat bladder 138 also preferably elevates the user slightly so that the user will rise away from the hard surface of housing 114. This may be facilitated by one or more gas bladders on the side of the liquid bladders away from the user's body.

For liquid bladders that need to “wrap around” a portion of a user's body or are otherwise required to be extended into place from an initial retracted position that allows ingress and egress of the user, a suitable mechanism is preferably provided to move and/or shape the bladder. For example, FIGS. 7A and 7B illustrate leg compression bladders 144a and 144b in deflated and inflated states, respectively. In this case, a suitably shaped gas bladder 148a and 148b is located adjacent to a respective liquid bladder 144a and 144b. Compressed air is injected into the air bladders 148a and 148b, causing them to each inflate into a desired configuration. The liquid bladders 144a and 144b are thus wrapped around the user's legs, as shown, so that essentially the entire circumference of the leg is contacted by the liquid bladder. The gas bladders also compensate for variances in the body sizes of various users.

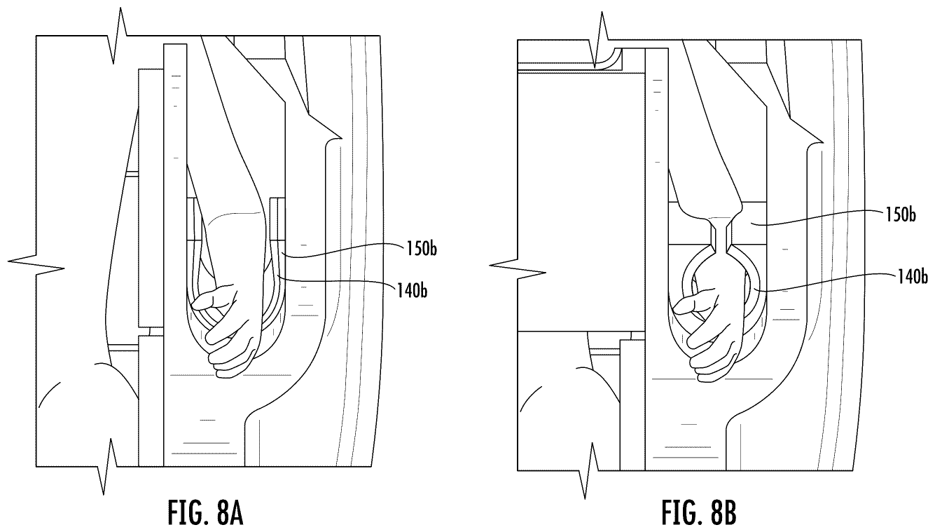

FIGS. 8A and 8B illustrate left arm compression bladder 140b in the deflated and inflated states, respectively. A gas bladder 150b, located adjacent to bladder 140b, inflates so that bladder 140b will wrap around the user's arm. Of course, the right arm compression bladder is similarly constructed with an associated gas bladder.

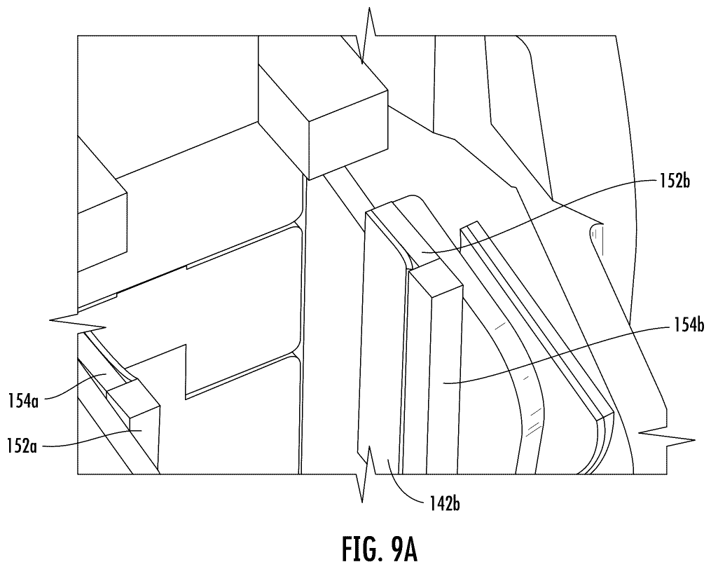

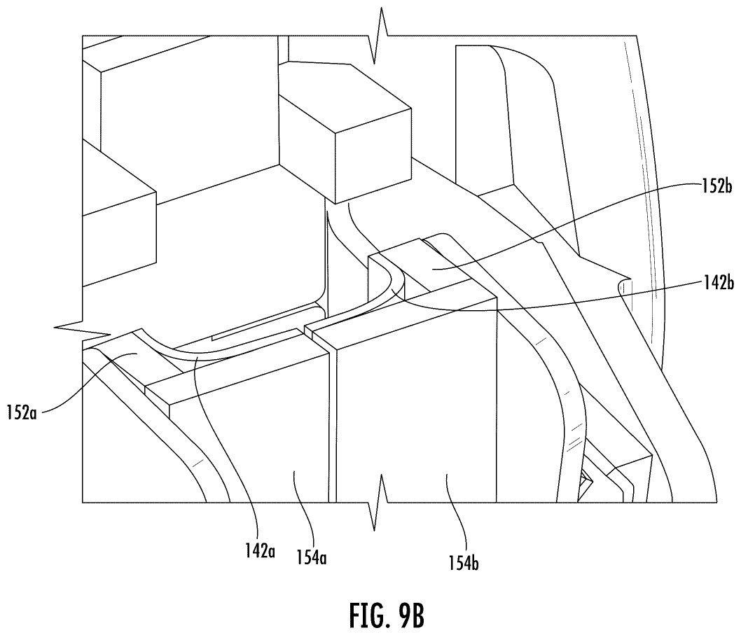

FIGS. 9A and 9B show side compression bladders 142a and 142b in deflated and inflated states, respectively. In this case, liquid bladders 142a and 142b respectively have a side gas bladder 152a-b and a top gas bladder 154a-b. The side gas bladders 152a-b expand and push the associated liquid bladder 142a-b against the user's side body. The top gas bladder 154a-b expands when inflated to push a position of the associated water bladder 142a-b against the front of the user's body. (The chest bladder may be optional depending on how much cold the user wants.) As shown, for example, in FIG. 3B, the side compression bladders 140 may have additional similar combinations of gas and liquid bladders (as indicated at 156a-b) that engage the sides and fronts of the user's thighs.

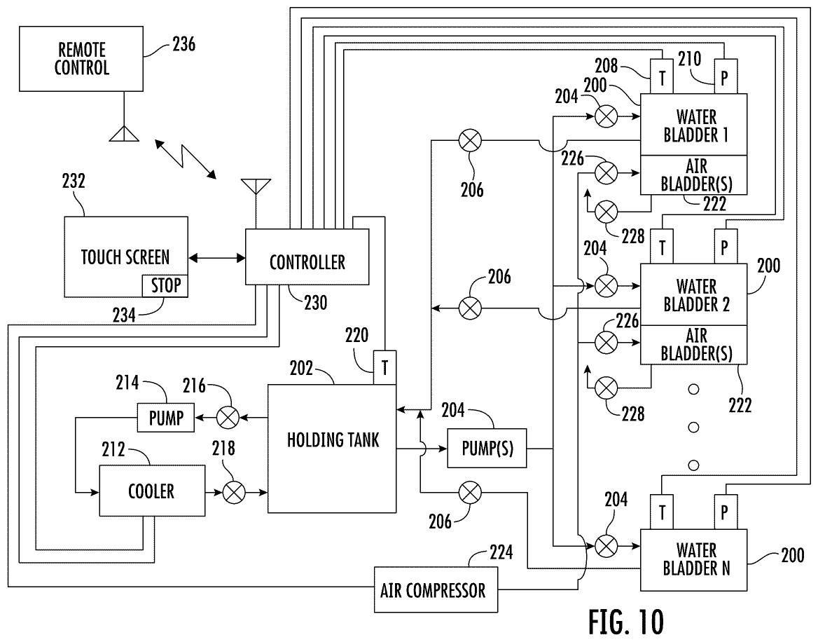

Referring now to FIG. 10, various components of an apparatus (such as those indicated at 10, 20, and 100 above) will be explained. One skilled in the art will be able to recognize which of the lines in FIG. 10 correspond to electrical signal lines and which of the lines correspond to piping to conduct the flow of fluid. As shown, a plurality of liquid bladders 200 (also indicated as water bladders 1, 2, . . . . N) are in fluid communication with a holding tank 202 containing a quantity of liquid chilled and maintained to the desired temperature. (A variety of liquids are appropriate for this application, but many preferred embodiments will simply use water as the liquid.) In this regard, one or more pumps 204 draw liquid from the tank and pass it along to the liquid bladders 200 so they will fill. Individual pumps may be used for each of the bladders or a smaller number of pumps may feed multiple bladders. Valves, such as those indicated 204 and 206, open to allow water to enter and exit a respective bladder. When closed, the valves 204 and 206 either contain a bladder in a filled state or isolate the associated bladder from the holding tank 202 (e.g., prevent water from entering the bladder). It will be appreciated that valves 204 and 206 may open partially during a cryotherapy session to allow exchange of warming water while also maintaining an appropriate amount of inflation. In this regard, at least one temperature sensor 208 and at least one pressure sensor 210 is preferably provided to ensure that the bladders are each correctly inflated and is maintaining the desired temperature. The sensors may be associated directly with the respective liquid bladders as shown, or may be located at appropriate places in the liquid circulation arrangement.

The temperature of the water in the holding tank 202 is maintained in this case by a water cooler 212. Although embodiments are contemplated in which water cooler 212 is located inside the holding tank 202, water cooler 212 is external to the holding tank 202 in the illustrated embodiment. Water is circulated through cooler 212 via a recirculation arrangement that includes a pump 214, along with isolation valves 216 and 218. With valves 216 and 218 open, pump 214 is activated to circulate water through the cooler 212. As one skilled in the art will appreciate, cooler 212 includes a suitable heat exchanger to remove heat from the liquid that passes through and/or over it. The temperature of the liquid is monitored via one or more suitable temperature sensors 220.

Gas bladders 222 are associated with at least some of the liquid bladders 200. For example, the respective liquid bladders may interpose an associated gas bladder so that the gas bladder, when inflated, acts as a cushion to separate the adjacent portion of the auser's body from the rigid support structure for a more pleasing experience. The gas bladders may also be used to position the liquid bladders as described above. The gas bladders 22 are inflated in this embodiment by any suitable source of compressed gas (e.g., air) such as an on-board air compressor 224. Inlet valves 226 open to provide fluid communication between the outlet of the air compressor 224 and the associated gas bladder 222. Exhaust valves 228, which are normally closed when the gas bladders are being or are inflated, open when it is desired to deflate the gas bladders 222. In the case or air, it may simply be exhausted to atmosphere, as shown.

A suitable controller 230 is provided to control the operation of various components of the dry cryotherapy apparatus. It will be appreciated that controller 230 will typically have one or more microprocessors and associated memory running appropriate program instructions. Controller 230 will also generally include appropriate analog-to-digital (A/D) converters and/or other interfaces to receive inputs from the various sensors and provide control signals to the various electromechanical components (pumps, valves, cooler 212, etc.). The apparatus may also have a user interface, here in the form of a touchscreen 232, to allow the user or other authorized person to control various settings as necessary or desired, such as water temperature, session duration, etc. In some cases, the user may be allowed to input physical characteristics such as height and weight to assist controller 230 in determining the extent to which the various bladders should be inflated (or filled). The user interface also preferably has a “stop” button or switch 234 so that the user can end a session at any desired time. The user interface may also be configured to provide entertainment, e.g., television shows, to the user during the session.

A suitable remote control 236 may also be provided. For example, remote control 236 may take the form of an “app” running on a mobile device, such as a mobile phone or tablet computer. The remote control 236 may communicate with the controller 230 via wired or wireless connection, as necessary or desired. In this case, a wireless connection, which may be via a wide area (e.g., the internet) or local area network (e.g., Bluetooth, Zigbee, etc.), is provided.

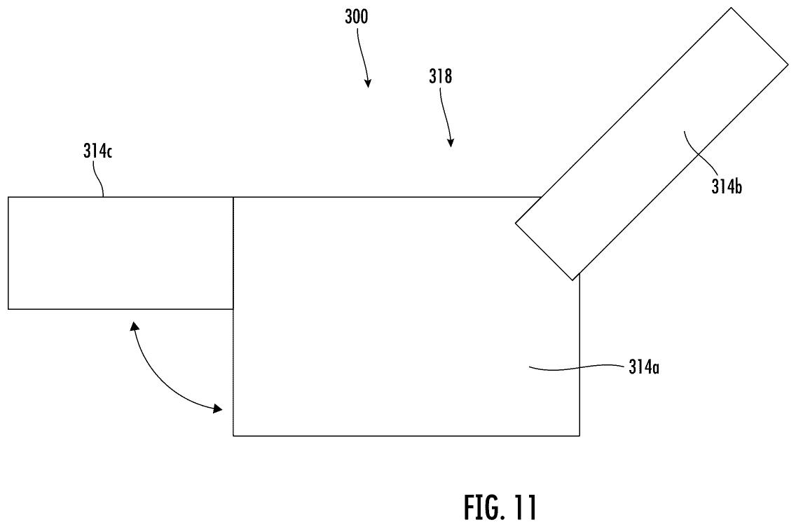

FIG. 11 shows a further embodiment of an apparatus 300 for providing dry cryotherapy in accordance with the present invention. A housing 314 defines a user receiving area (generally 318). Consistent with the above description, apparatus 300 utilizes a series of water and gas bladders to support and/or envelop the user with cold fluid during the session. In this case, the user will be supported in a reclined position during the dry cryotherapy session.

Toward this end, housing 314 includes a main (or seat) portion 314a where the buttocks and thighs of the user's body will be located. A back portion 314b extends away from the main portion 314a at an oblique angle for supporting the user's upper body. Finally, a leg portion 314c supports the user's legs below the knees. In use, leg portion 314c extends away from main portion 314a, as shown. When not in use, however, leg portion 314c will retract (as indicated by the arrow) to a vertical orientation. This makes it easier for the user to get on and off the unit. Back portion 314b may also recline as necessary or desired.

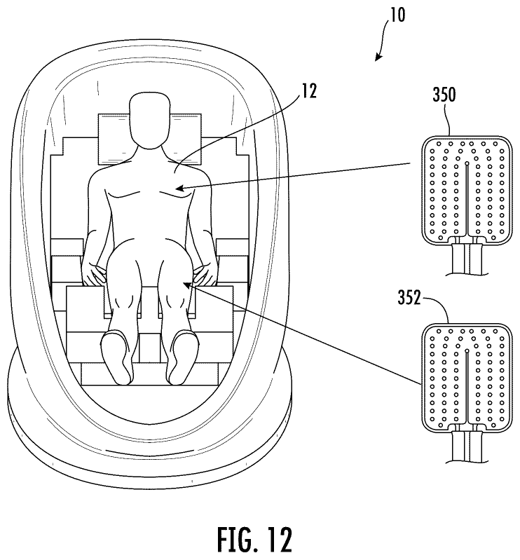

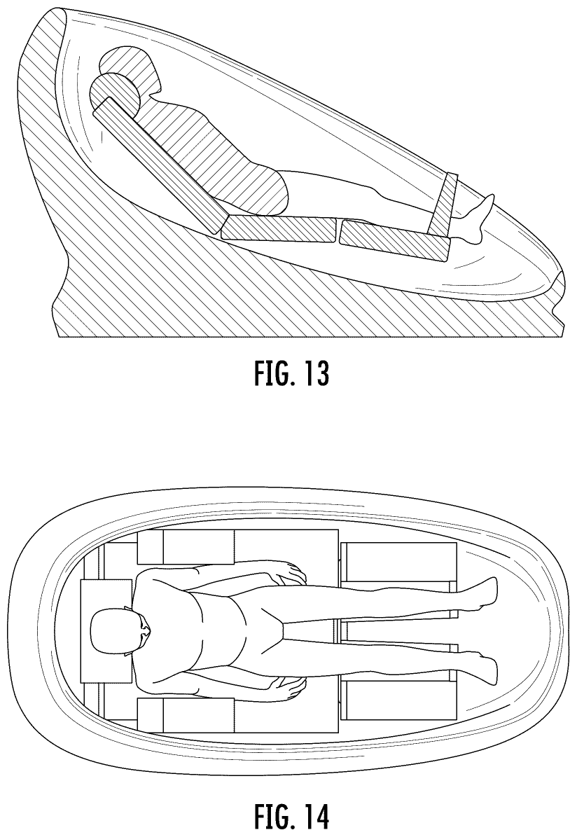

FIGS. 12-14 show additional views of the apparatus 12. As shown in FIG. 12, optional cold therapy pads, such as those indicated at 350 and 352, may be provided for placement on selected areas of the user's body, such as the chest and top of legs. The pads may be selectively filled and evacuated of liquid as described above.

It can thus be seen that the present invention provides novel apparatus for achieving dry cryotherapy. Methods of achieving dry cryotherapy are also provided. Aspects of the present invention provide an experience very similar to full body cold immersion, so the user receives the benefits of a cold plunge. But the experience can be achieved in a more innovative, advanced form. For example, the user stays clothed and dry during the session, which is an advantage in comparison with traditional cold-water immersion. Also, the user does not come into contact with sweat and other contaminants that may be present in a small pool of water through immersion by a prior user.

While one or more preferred embodiments of the invention are described above, it should be appreciated by those skilled in the art that various modifications and variations can be made in the present invention without departing from the scope and spirit thereof.

What is claimed is:

1. A dry cryotherapy apparatus for use by a user, the apparatus comprising:
a support structure defining a user receiving area at which the user will be located for dry cryotherapy;
a plurality of liquid bladders located in the user receiving area, the liquid bladders configured to contain a quantity of liquid;
a reservoir operative to contain a quantity of the liquid, the reservoir being in selective fluid communication with the plurality of bladders via a liquid circulation arrangement; and
a cooler operative to cool the liquid in the reservoir to a selected temperature,
wherein supply of the liquid from the reservoir to one of the liquid bladders causes the bladder to fill such that an adjacent portion of the user's body will be cooled by the liquid.

2. The dry cryotherapy apparatus of claim 1, wherein the cooler is external to the reservoir such that the liquid being cooled circulates from the reservoir through the cooler and back to the reservoir.

3. The dry cryotherapy apparatus of claim 1, further comprising at least one pump to supply the cooled liquid to the liquid bladders.

4. The dry cryotherapy apparatus of claim 3, further comprising at least one temperature sensor and at least one pressure sensor.

5. The dry cryotherapy apparatus of claim 4, wherein the reservoir comprises a water tank.

6. The dry cryotherapy device of claim 4, further comprising a plurality of electromechanical valves situated in the liquid circulation arrangement.

7. The dry cryotherapy device of claim 6, further comprising a controller programmed to effect operation of the at least one pump and the electromechanical valves based at least in part on data from the at least one temperature sensor and the at least one pressure sensor.

8. The dry cryotherapy apparatus of claim 1, further comprising a movement mechanism associated with at least some of the liquid bladders, the movement mechanism being operative to cause an associated liquid bladder to cover a front part of the adjacent portion of the user's body.

9. The dry cryotherapy apparatus of claim 8, wherein the movement mechanism comprises a gas bladder associated with the associated liquid bladder.

10. The dry cryotherapy apparatus of claim 9, further comprising a compressed air source operative to inflate the gas bladder.

11. The dry cryotherapy apparatus of claim 1, wherein the plurality of liquid bladders comprises at least four bladders.

12. The dry cryotherapy apparatus of claim 1, wherein the plurality of liquid bladders comprises at least five bladders.

13. The dry cryotherapy apparatus of claim 1, wherein the plurality of liquid bladders comprises at least six bladders.

14. The dry cryotherapy apparatus of claim 1, wherein the plurality of liquid bladders comprises at least one shoulder bladder, at least one back bladder, at least one seat bladder, a pair of arm compression bladders, a pair of side compression bladders, and a pair of leg compression bladders.

15. The dry cryotherapy apparatus of claim 1, further comprising a user interface.

16. The dry cryotherapy apparatus of claim 15, wherein the user interface comprises a touch screen.

17. The dry cryotherapy apparatus of claim 1, wherein the user receiving area is configured such that the user will be in a semi-reclined position.

18. A dry cryotherapy apparatus for use by a user, the apparatus comprising:
a support structure defining a user receiving area at which the user will be located for dry cryotherapy;
a user interface;
a plurality of liquid bladders located in the user receiving area, the liquid bladders configured to contain a quantity of liquid;
a reservoir operative to contain a quantity of the liquid, the reservoir being in selective fluid communication with the plurality of bladders via a liquid circulation arrangement;
at least one pump located in the liquid circulation arrangement to supply the liquid from the reservoir to the liquid bladders;
a plurality of electromechanical valves situated in the liquid circulation arrangement; and
a cooler operative to cool the liquid in the reservoir to a selected temperature,
wherein supply of the liquid from the reservoir to one of the liquid bladders causes the bladder to be in therapeutic engagement with an adjacent portion of the user's body.

19. A method of performing dry cryotherapy, the method comprising steps of:
providing an apparatus having:
a support structure defining a user receiving area at which the user will be located for dry cryotherapy; and
a plurality of liquid bladders located in the user receiving area, the liquid bladders being in a deflated state;
providing a source of cold water; and
supplying a quantity of the cold water to the plurality of liquid bladders so that the bladders will be in an inflated state; and
causing the quantity of water to leave the bladders so that the bladders are again in the deflated state.

20. The method of claim 19, wherein the step of providing a supply of cold water involves chilling the water to a temperature of between approximately 33 to 45 degrees Fahrenheit.

21. The method of claim 19, wherein the bladders are maintained in the inflated state for a predetermined period of time between approximately 8-10 minutes.
